(19) United States
(12) Patent Application Publication
(10) Pub. No.: US 2026/0295187 A1
(43) Pub. Date: Oct. 1, 2026

(54) A DIFFICULT AIRWAY VENTILATION DEVICE

(71) Applicant: Peking University Third Hospital (The Third Clinical Medical School of Peking University), Beijing (CN)

(72) Inventors: Mao XU, Beijing (CN); Bin WEI, Beijing (CN); Tianhao ZHANG, Beijing (CN); Yujie WANG, Beijing (CN); Rui ZHANG, Beijing (CN)

(73) Assignee: Peking University Third Hospital (The Third Clinical Medical School of Peking University), Beijing (CN)

(21) Appl. No.: 18/996,580

(22) PCT Filed: May 15, 2023
(86) PCT No.: PCT/CN2023/094124

§ 371 Date: Jan. 17, 2025

(30) Foreign Application Priority Data
Jul. 18, 2022 (CN) 202221833662.3

(51) Int. Cl. A61M 16/04; A61B 1/267

(52) U.S. Cl. CPC A61M 16/0488; A61B 1/267; A61M 16/0465

(57) ABSTRACT

An airway ventilation device, includes an airway stent assembly and a guidance assembly, and the guidance assembly is used for guiding the airway stent assembly to be placed in the supraglottis position. The airway stent assembly includes an inner tube, an outer tube and a locking unit, the outer tube is sleeved on the inner tube, the locking unit is arranged on the inner tube and the outer tube, and the locking unit is used for locking the inner tube and the outer tube; and the airway stent assembly can be used as a device for guaranteeing ventilation and auxiliary tracheal intubation.

### CROSS-REFERENCE TO RELATED APPLICATIONS

This application is a National Stage of International Application No. PCT/CN 2023/094124, filed on May 15, 2023, which claims priority to Chinese Patent Application No. 202221833662.3, filed on Jul. 18, 2022. The above-referenced application is hereby incorporated by reference in their entirety.

### TECHNICAL FIELD

The present application relates to the technical field of medical device, and particularly relates to a difficult airway ventilation device.

### BACKGROUND

Safe airway management is an important measure to ensure the safety of patients during the perioperative period, and it is one of the most challenging tasks in the perioperative period. Difficult airway often occurs in clinical work, and proper airway management is related to patient safety and directly affects patient prognosis and outcome.

Difficult airway manifest as clinical conditions such as difficulty in ventilation or intubation, or both. Difficult ventilation is mostly caused by obstruction of tissues at different levels of the upper respiratory tract, while difficult intubation is caused by difficulty in glottal exposure or the need for multiple attempts in intubation. Patients with a non-difficult airway may also evolve into an urgent difficult airway due to inappropriate clinical management, which affects the accuracy of airway assessment methods in predicting difficult airway and the effectiveness of airway management.

At present, the techniques and tools to effectively deal with difficult airway in clinical work are still not perfect, and the existing technologies and tools are often unable to effectively deal with difficult airway, especially emergency airway that cannot be ventilated or intubated, due to factors such as exposure of visual field, space, anatomical abnormalities and secretions, which may seriously endanger patients' lives and bring severe challenges to airway management. There is an urgent need to provide new solutions for difficult airway, especially emergency airway, and simplify the treatment process of difficult airway, so as to overcome the limitations of current difficult airway management techniques and simultaneously solve the core problems of “effective ventilation” and “safe intubation”, which will also have a profound impact on difficult airway management.

Therefore, the development of new difficult airway management tools, improving the safety and effectiveness of airway management, and simplifying the treatment process of difficult airway, especially emergency airway, are major clinical problems that need to be solved urgently.

### SUMMARY

In view of the above analysis, the embodiment of the present disclosure is intended to provide a difficult airway ventilation device to solve the problem of ineffective ventilation caused by obstruction in difficult airway.

The object of the present disclosure is mainly realized by the following technical scheme:

a difficult airway ventilation device comprising a guidance assembly and an airway stent assembly, the guidance assembly is used to guide the airway stent assembly; the guidance assembly comprises an air bag, and the air bag is used to guide the airway stent assembly; the airway stent assembly comprises an inner tube, an outer tube and a locking unit, the outer tube is sleeved on the inner tube, the locking unit is arranged on the inner tube and the outer tube, the locking unit is used for locking the inner tube and the outer tube, and the airway stent assembly can be used as a path for ventilation and tracheal intubation.

Further, the airbag is provided with a tube, and the tube inflates and deflates the airbag.

Further, the airbag is provided with a first through hole, the flexible intubation scope passes through the first through hole, and the airbag can be fixedly connected with the flexible intubation scope through the first through hole.

Further, an anti-slip layer is arranged on the inner wall of the first through hole. Further, the airbag is spindle-shaped after inflation, and the airbag comprises a central axis, and the first through hole is coaxial with the airbag.

Further, the guidance assembly further comprises a guidewire and the airbag is provided with a second through hole, through which the guidewire is connected to the airbag movably and is used to guide the flexible intubation scope and the airbag into the difficult airway.

Further, the guidewire comprises a bending portion, which is arranged at one end of the guidewire, and the bending portion can be bent at will and maintain the shape after bending.

Further, one end of the outer tube is provided with an outer flange, the outer flange protrudes from the outer wall of the outer tube, the locking unit comprises an inner flange, the inner flange is arranged on the inner wall of the locking unit, the inner diameter of the inner flange is smaller than the outer diameter of the outer flange, and the outer flange and the inner flange cooperate with each other to limit the movement of the locking unit.

Further, the locking unit further comprises a groove, the inner tube comprises a convex ridge, the convex ridge is arranged at one end of the inner tube, and the groove and the convex ridge are clamped.

Further, the length of the stent is retractable and the inner diameter can be adjusted.

Further, the stent comprises a supporting body, a retrievable unit and a tail bracing unit, the retrievable unit is arranged at one end of the supporting body, and the tail bracing unit is arranged at the other end of the supporting body; the tail bracing unit forms a hollow blade-shaped.

Further, the retrievable unit comprises a first support rod, a second support rod, a tail ring and a drawbar, the number of the second support rod is two, the first support rod is located between the two second support rods, one end of the two second support rods and one end of the first support rod are all connected with the tail ring, and the drawbar is connected with the tail ring for retrieving the stent.

Compared with existing technology, the present application achieves at least one of the following beneficial effects:
1. The stent of the difficult airway ventilation device of the present disclosure can quickly relieve the upper airway obstruction and ensure ventilation in difficult airway condition;
2. The tail bracing unit of the stent of the present disclosure can expose the airway and larynx, facilitate the observation of the airway and the glottis, assist intubation, and also establish an effective suction channel;
3. The retrievable unit of the stent of the present disclosure makes the stent convenient to remove, and reduces the damage to the patient's oral cavity and airway;
4. The airbag of the guidance device provided by the present disclosure can guide the outer tube to be placed in the supraglottis position, and the flexible intubation scope is coaxial with the airbag, so that in the process of guiding the airway stent assembly, the airway stent assembly is coaxial with the flexible intubation scope, and the airway stent assembly no longer deviates from the axis of the flexible intubation scope, which greatly facilitates the guidance of the flexible intubation scope to the airway stent assembly;
5. The recessed portion of the present disclosure reduces the contact area between the air bag and the airway in the process of guiding the external tube, and can maintain airway ventilation;
6. The guidewire of the present disclosure is used to guide the flexible intubation scope, and the bending portion makes one end of the guidewire have a larger area when advancing in the larynx, and when the guidewire touches the larynx or the inner wall of the trachea, the pressure on the larynx or the inner wall of the trachea is reduced, and the injury to the larynx or trachea is reduced.

### BRIEF DESCRIPTION OF THE DRAWINGS

The drawings are only for the purpose of showing specific embodiments, and are not considered to limit the present application. In the whole drawing, the same reference symbols represent the same components.

FIG. 1 is a schematic diagram of the overall structure of the ventilation device in embodiment 1;

FIG. 2 is a schematic diagram of the internal structure of the ventilation device in embodiment 1;

FIG. 3 is a schematic diagram of the structure of the release state of the stent in embodiment 1;

FIG. 4 is a schematic diagram of the overall structure of the ventilation device in embodiment 2;

FIG. 5 is a schematic diagram of the internal structure of the ventilation device in embodiment 2;

FIG. 6 is a schematic diagram of the structure of the release state of the stent in embodiment 2;

FIG. 7 is a schematic diagram of the overall structure of the guidance assembly in embodiment 2;

FIG. 8 is a schematic diagram of the use state of the guidance component in embodiment 2.

### REFERENCE NUMERALS ARE AS FOLLOWS

### Embodiment 1

1—inner tube; 11—convex ridge; 2—outer tube; 21—outer flange; 3—stent; 31—first opening; 32—second opening; 4—retrievable mechanism; 41—first support rod; 42—second support rod; 43—tail ring; 44—drawbar; 5—skeleton; 6—locking portion; 61—inner flange; 62—groove; 7—indicator line.

### Embodiment 2

1—inner tube; 2—outer tube; 3—locking unit; 4—stent; 5—indicator line; 6—airbag; 7—guidewire; 8—flexible intubation scope; 11—ventilation hole; 12—convex ridge; 21—outer flange; 31—inner flange; 32—groove; 41—supporting body; 42—retrievable unit; 43—tail bracing unit; 61—tube; 62—first through hole; 71—bending portion; 411—first opening; 412—second opening; 421—first support rod; 422—second support rod; 423—tail ring; 424—drawbar.

### DESCRIPTION OF EMBODIMENTS

The preferred embodiment of the present disclosure is described below in conjunction with the accompanying drawings, wherein the accompanying drawings form a part of the present disclosure and are used together with the embodiments of the present disclosure to illustrate the principle of the present disclosure and are not used to limit the scope of the present disclosure.

### Embodiment 1

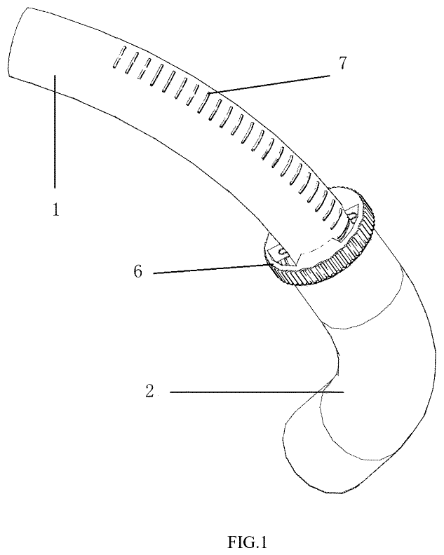

As shown in FIG. 1-FIG. 3, an airway ventilation device is disclosed, which comprises an inner tube 1, an outer tube 2, a stent 3, a retrievable mechanism 4 and a skeleton 5, the outer tube 2 is arranged at one end of the inner tube 1; the stent 3 is arranged in the outer tube 2, one end of the stent 3 is provided with the retrievable mechanism 4, and the other end of the stent 3 is provided with the skeleton 5.

Preferably, the stent 3 is used for the opening of the airway and keeping it open.

Preferably, the inner tube 1 has an arc adapted to the human airway, the inner tube 1 is a round tube, and further, the inner tube 1 is a rigid round tube, and the inner tube 1 provides guidance and support for the outer tube 2.

Preferably, the outer tube 2 is a round tube; and the outer tube 2 has the same arc as the inner tube 1. The outer tube 2 can open the difficult airway during endotracheal intubation, and the combination of the inner tube 1 and the outer tube 2 can be used as an endotracheal tube; The outer tube 2 is capable of sliding along the inner tube 1.

Preferably, the stent 3 is an elastic support to fit the shape in the airway.

Preferably, the stent 3 is composed of a grid structure formed by a plurality of elastic fiber spirally braided, and the length of the stent 3 is retractable and the inner diameter can be adjusted.

The lower end of the outer tube 2 is convex or flush relative to the lower end of the stent 3, and the stent 3 is constrained by the outer tube 2 and is in a radial shrinkage state.

Preferably, the outer tube 2 is a conical tube, and the diameter of the bottom end of the outer tube 2 is smaller than the diameter of the top end of the outer tube 2, so as to facilitate the insertion of the difficult airway of the outer tube 2.

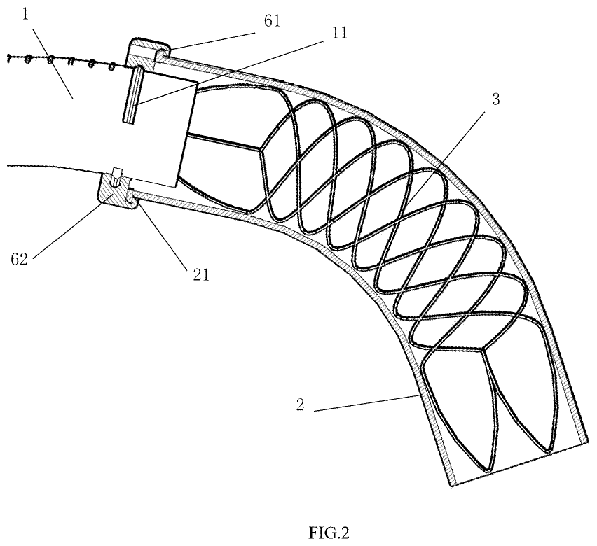

Preferably, see FIG. 1-FIG. 2, the upper end of the outer tube 2 is provided with an outer flange 21, and the outer flange 21 protrudes from the outer wall of the outer tube 2.

Preferably, the outer flange 21 is provided with a locking portion 6, the locking portion 6 is provided with an inner flange 61, the inner flange 61 protrudes from the inner wall of the locking portion 6, the inner diameter of the inner flange 61 is smaller than the outer diameter of the outer flange 21, and the outer flange 21 and the inner flange 61 cooperate with each other to limit the movement of the locking portion 6 and ensure that the locking portion 6 will not be separated from the outer tube 2.

Preferably, the locking portion 6 is further provided with a groove 62, the lower end of the inner tube 1 is also provided with a convex ridge 11, the groove 62 and the convex ridge 11 can be clamped with each other, the relative positions of the outer tube 2 and the inner tube 1 are locked through the locking portion 6, the outer tube 2 is prevented from sliding on the inner tube 1 during intubation operation, and the stent 3 is ensured that the stent 3 will not be released in advance during the tracheal intubation operation.

Preferably, an indicator line 7 is arranged on the outer wall of the inner tube 1, and the insertion depth information of the outer tube 2 is convenient for reading when the placement operation of the stent 3 is implemented.

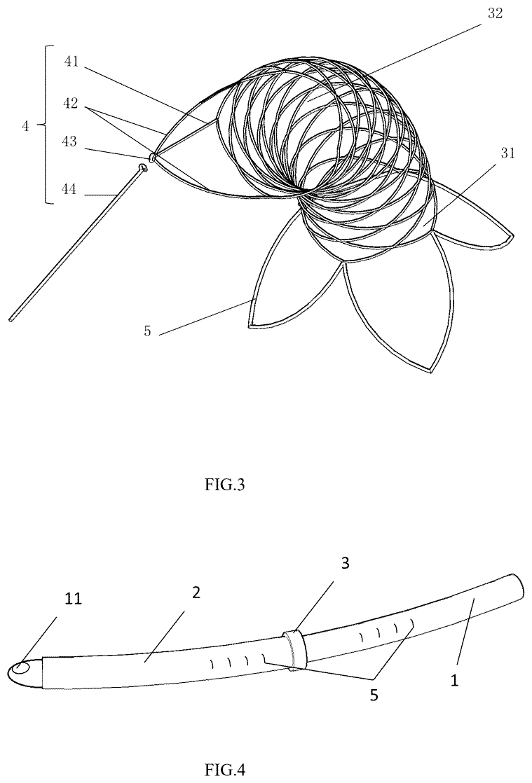

Preferably, the stent 3 forms the first opening 31 and the second opening 32, see FIG. 3, after the stent 3 is released in the airway, the first opening 31 is in the glottis position, and the second opening 32 is in the oral cavity.

After the stent 3 is released, it is against the airway, and the gap of the grid structure can expose the airway and the larynx, so that the airway and the larynx can be easily observed, sampled or treated.

Preferably, the upper end of the stent 3 is provided with a retrievable mechanism 4, see FIG. 3 for details, and the retrievable mechanism 4 can be used to retrieve the stent 3, and can also support the oral cavity to keep the second opening 32 open.

Preferably, the retrievable mechanism 4 comprises a first support rod 41, a second support rod 42, a tail ring 43 and a drawbar 44.

Preferably, of the number of the second support rod 42 is two, the first support rods 41 are located between the two second support rods 42, and one end of each of the two second support rods 42 and one end of the first support rods 41 are all connected with the tail ring 43.

Preferably, the drawbar 44 is connected with the tail ring 43 to retrieve the stent 3.

Preferably, a skeleton 5 is arranged at the lower end of the stent 3, the skeleton 5 is woven by elastic fibers, the skeleton 5 can be bounced open to resist the upper part of the throat, the stent 3 is prevented from sliding down to the throat, and the first opening 31 is opened. Skeleton 5 forms a hollow blade shape so that the airway area of skeleton 5 is exposed, so that the airway can be observed, sampled, or treated.

When in use, the open end of the outer tube 2 is extended into the oral cavity, and after it is in place, the position of the inner tube 1 is maintained, the locking portion 6 is rotated until the groove 62 of the locking portion 6 and the convex ridge 11 on the inner tube 1 are completely separated, the locking portion 6 is pulled upward, the locking portion 6 drives the outer tube 2 to be withdrawn out of the oral cavity along the inner tube 1, and the stent 3 is released simultaneously until the stent 3 is fully deployed in the airway. At this point, the first opening 31 is in the glottis position, and the second opening 32 is in the oral cavity.

See FIG. 2, when the stent 3 is not released, the skeleton 5 is in a retracted state, because it is restrained by the outer tube 2 and is in a radial contraction state. The retrievable mechanism 4 is between the inner tube 1 and the outer tube 2; After the stent 3 is released, see FIG. 3, and the skeleton 5 is bounced open in a trumpet shape.

When the stent 3 is removed from the airway, the drawbar 44 is first hooked to the tail ring 43, and then the drawbar 44 is penetrated from the bottom end of the outer tube 2 and is threaded out from the top end. At the same time the drawbar 44 is pulled outward from the oral cavity, the outer tube 2 is inserted into the oral cavity, and the drawbar 44 pulls the tail ring 43 and then pulls the first support rod 41 and the second support rod 42 until the stent 3 is completely included in the outer tube 2.

The inner tube 1 is locked with the outer tube 2 through the locking part 6, and then the entire airway ventilation device is sterilized so that it can be used again.

Compared with the prior art, the airway ventilation device provided in this embodiment can quickly relieve the obstruction of the upper airway when the airway condition is difficult, establish a spacious endotracheal intubation path, can also establish an effective suction channel, can also guide the endoscope to effectively determine the anatomical position where it is located, and can be used repeatedly, reduce medical costs, reduce the burden of patients, reduce the probability of emergency surgical airway, and is more conducive to patient safety.

### Embodiment 2

A specific embodiment of the present disclosure, as shown in FIG. 4, discloses a difficult airway ventilation device (hereinafter referred to as the ventilation device), which comprises an airway stent assembly and a guidance assembly, and the guidance assembly is used to guide the airway stent assembly to be placed in the supraglottis position.

Preferably, as shown in FIG. 4, the airway stent assembly comprises an inner tube 1, an outer tube 2 and a locking unit 3, the outer tube 2 is sleeved on the inner tube 1, the locking unit 3 is arranged on the inner tube 1 and the outer tube 2, and the locking unit 3 is used for locking the inner tube 1 and the outer tube 2. The relative positions of the outer tube 2 and the inner tube 1 are locked by the locking unit 3 to prevent the outer tube 2 from sliding on the inner tube 1 during intubation operation. The airway stent assembly is designed to open a difficult airway that has narrowed, keep the patient breathing open, and can also be used as an aid to endotracheal intubation.

Preferably, the inner tube 1 has an arc adapted to the human airway, the inner tube 1 is a round tube, and further, the inner tube 1 is a rigid round tube, and the inner tube 1 provides guidance and support for the outer tube 2.

Preferably, one end of the inner tube 1 protrudes from the end face of the outer tube 2, and a ventilation hole 11 is arranged on the side wall of the end of the inner tube 1 protruding from the outer tube 2, so that the two ends of the inner tube 1 are kept ventilated, and are used to keep the airway open during airway intubation. One end of the inner tube 1 protruding from the outer tube 2 is droplet-shaped, and the airway stent assembly is inserted into the airway to avoid causing damage to the airway.

Preferably, the outer tube 2 is a round tube; the outer tube 2 has the same arc as the inner tube 1; the outer tube 2 can enter the difficult airway, and the combination of the inner tube 1 and the outer tube 2 can be used as an endotracheal tube; the outer tube 2 is capable of sliding along the inner tube 1.

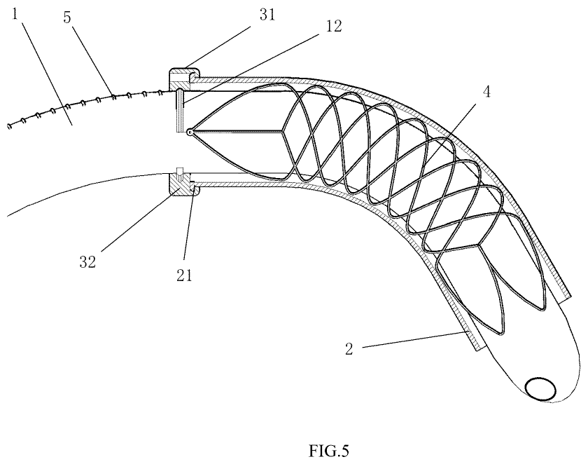

Preferably, as shown in FIG. 5, one end of the outer tube 2 is provided with an outer flange 21, the outer flange 21 protrudes from the outer wall of the outer tube 2, the locking unit 3 is arranged on the outer flange 21, the locking unit 3 comprises an inner flange 31, the inner flange 31 is arranged on the inner wall of the locking unit 3, the inner diameter of the inner flange 31 is smaller than the outer diameter of the outer flange 21, the outer flange 21 and the inner flange 31 cooperate with each other, limit the movement of the locking unit 3, and ensure that the locking unit 3 will not be separated from the outer tube 2.

Preferably, the locking unit 3 further comprises a groove 32, the inner tube 1 comprises a convex ridge 12, the convex ridge 12 is arranged at one end of the inner tube 1, and the groove 32 and the convex ridge 12 are clamped with each other, so that the locking unit 3 can lock the relative positions of the outer tube 2 and the inner tube 1, and prevents the outer tube 2 from sliding on the inner tube 1 during the intubation operation.

Preferably, the ventilation device of the present disclosure further comprises a stent 4, the stent 4 is arranged in the outer tube 2 in a contracted state, and the stent 4 is used for the opening of the difficult airway and keeps the difficult airway open.

Preferably, the stent 4 is an elastic support to accommodate the shape in the airway.

Preferably, the lower end of the outer tube 2 is convex or flush relative to the lower end of the stent 4, and the stent 4 is constrained by the outer tube 2 and is in a radial contraction state.

Preferably, the stent 4 is composed of a grid structure formed by a plurality of elastic material fibers spirally braided, and the length of the stent 4 is retractable and the inner diameter can be adjusted. The stent 4 can be released, and after release, the stent 4 can resist and support the airway, increase the air circulation in the airway, and the gap of the grid structure can expose the airway and the larynx, realize ventilation, facilitate the observation of the airway and the larynx, assist intubation, and also establish an effective suction channel, and can quickly draw the secretions or blood in the patient's oral cavity.

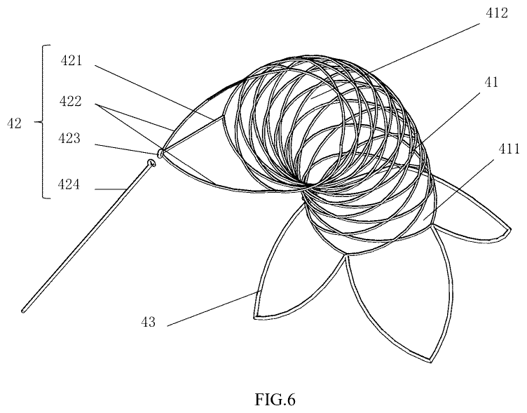

Preferably, as shown in FIG. 6, the stent 4 comprises a supporting body 41, a retrievable unit 42 and a tail bracing unit 43, the retrievable unit 42 is arranged at one end of the supporting body 41, and the tail bracing unit 43 is arranged at the other end of the supporting body 41.

Preferably, the two ends of the supporting body 41 are opened, respectively, the first opening 411 and the second opening 412, and after the stent 4 is released in the airway, the first opening 411 is in the supraglottic laryngopharyngeal position, and the second opening 412 is in the oropharynx.

Preferably, the supporting body 41 is a self-expanding elastic structure, the supporting body 41 can be provided with a covering film, the covering film wraps the outer wall of the supporting body 41, and the covering film ensures the overall support of the supporting frame 4 to the larynx, so as to reduce the extrusion of the larynx by the grid structure.

Preferably, the retrievable unit 42 comprises a first support rod 421, a second support rod 422, a tail ring 423 and a drawbar 424. The retrievable unit 42 can be used for the retrieval of stent 4 and can also support the oral cavity to keep the second opening 412 open.

Preferably, the number of the second support rod 422 is two, the first support rods 421 are located between the two second support rods 422, and one end of the two second support rods 422 and one end of the first support rods 421 are connected with the tail ring 423.

Preferably, the drawbar 424 is connected with the tail ring 423 to retrieve the stent 4.

The retrievable unit 42 makes the stent 4 conveniently removed, and reduces the damage to the patient's oral cavity and airway.

Preferably, the tail bracing unit 43 is woven of elastic material fibers, and the tail bracing unit 43 can be bounced open to resist the surrounding tissues on the glottis and expose the glottis, so that the first opening 411 remains open. The tail bracing unit 43 forms a hollow blade shape so that the airway area opened by the tail bracing unit 43 is exposed and the airway is convenient for ventilation, observation, assisted intubation, sampling, or treatment.

Preferably, the ventilation device of the present disclosure further comprises an indicator line 5, the indicator line 5 is arranged on the outer wall of the inner tube 1 and the outer tube 2, and the depth information of the insertion of the outer tube 2 into the oral cavity is convenient when the placement operation of the stent 4 is implemented.

Because the first placement of the stent 4 is placed under blind probe, there may be the problem that the placement position is not optimal, and the guidance assembly can be used to adjust the placement position of the ventilation device for the second combined flexible intubation scope 8.

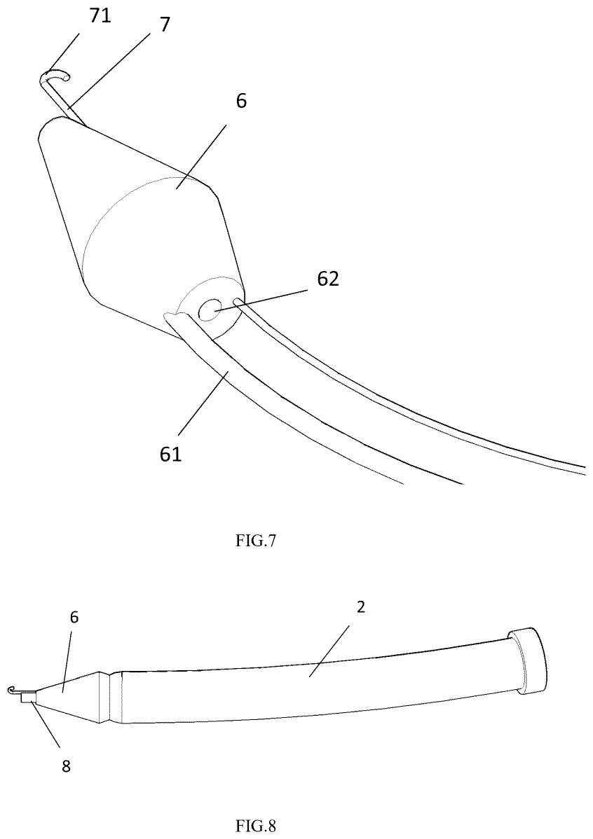

Preferably, as shown in FIG. 7 and FIG. 8, the guidance assembly comprises an airbag 6, and the airbag 6 is spindle-shaped. The airbag 6 can be inflated and deflated, and one end of the airbag 6 can fill one end of the outer tube 2 after being inflated, and is connected with the outer tube 2. The airbag 6 is provided with a tube 61, and the tube 61 is inflated and deflated for the airbag 6. One end of the airbag 6 can be inserted into the obstructed upper airway to guide the outer tube 2 to be placed in the supraglottis position.

Preferably, the airbag 6 is provided with a first through hole 62, the flexible intubation scope 8 passes through the first through hole 62, and the airbag 6 can be fixedly connected with the flexible intubation scope 8, and then the outer tube 2 is guided to be placed in the supraglottis position.

Preferably, an anti-slip layer is arranged on the inner wall of the first through hole 62, and in the inflated state of the airbag 6, the anti-slip layer increases the friction force to ensure that the airbag 6 and the flexible intubation scope 8 do not displace.

Preferably, the airbag 6 comprises a central axis, the first through hole 62 is coaxial with the airbag 6, the flexible intubation scope 8 is coaxial with the airbag 6, and the maximum outer diameter of the airbag 6 is equal to the inner diameter of the airway stent assembly, so that in the process of guiding the airway stent assembly, the airway stent assembly is coaxial with the flexible intubation scope 8, and the airway stent assembly no longer deviates from the axis of the flexible intubation scope 8, so as to facilitate the guidance of the flexible intubation scope 8 to the airway stent assembly.

Preferably, there is a recessed portion (not shown in the Figure) on the airbag 6, the recessed portion is parallel to the central axis, and the recessed portion in the process of guiding the outer tube 2 makes the airway 6 not completely block the airway and maintain difficult airway ventilation.

Preferably, the guidance assembly further comprises a guidewire 7, and the guidewire 7 is movably connected with the airbag 6. The guidewire 7 is used to guide the flexible intubation scope 8 into the glottis.

Preferably, the airbag 6 is also provided with a second through hole (not shown in the Figure), and the guidewire 7 is movably connected with the airbag 6 through the second through hole.

Preferably, the guidewire 7 comprises a bending portion 71, the bending portion 71 is arranged at one end of the guidewire 7, and the bending portion 71 can be bent at will and maintains the shape after bending. The bending portion 71 makes one end of the guidewire 7 have a larger area when advancing in the larynx, and when the guidewire 7 touches the larynx or the inner wall of the trachea, the pressure on the larynx or the inner wall of the trachea is reduced, and the injury to the larynx or trachea is reduced, and the patient safety is ensured.

In difficult airway conditions, especially emergency airway, the airway stent assembly is inserted into the difficult airway to relieve the airway of obstruction; keep the position of the inner tube 1 unchanged, rotate the locking unit 3 until the groove 32 of the locking unit 3 and the convex ridge 12 on the inner tube 1 are completely separated. When the stent 4 is not released, it is in a radial contraction state because it is restrained by the outer tube 2, and the tail bracing unit 43 is in a retracted state. The retrievable unit 42 is between the inner tube 1 and the outer tube 2; pull up the locking unit 3, the locking unit 3 drives the outer tube 2 to pull out along the inner tube 1 to the outside of the oral cavity, and the stent 4 is released simultaneously until the stent 4 is fully deployed in the airway, and after the stent 4 is released, the tail bracing unit 43 is bounced open in a flare-shaped shape. The stent 4 further stretches the airway to increase the air circulation in the airway. At this point, the first opening 411 is in the supraglottic laryngopharyngeal position, and the second opening 412 is in the oropharynx.

After the airway is opened, the placement position of the stent 4 is explored, if the placement position is not optimal, the outer tube 2 needs to be inserted again in the airway and the stent 4 is retrieved, the drawbar 424 is hooked to the tail ring 423, and the drawbar 424 is penetrated from the bottom end of the outer tube 2 and threaded out from the top end; while pulling the drawbar 424 outward from the oral cavity, the outer tube 2 is inserted into the oral cavity, and the drawbar 424 pulls the tail ring 423 and then pulls the first support rod 421 and the second support rod 422 until the stent 4 is completely included in the outer tube 2; then the guidance assembly is inserted into one end of the outer tube 2 to be inflated, and connected with the outer tube 2, the airway stent assembly is repositioned under the assisted photopic of the flexible intubation scope 8, the guidance assembly is withdrawn, the inner tube 1 is put back into the outer tube 2 for preventing the stent 4 from being withdrawn with the outer tube 2, then the inner tube 1 is not moved, the outer tube 2 is returned to release the stent 4 again, and the inner tube 1 is withdrawn again.

Specifically, the airbag 6 is sleeved on the flexible intubation scope 8 through the first through hole 62 and inflated. The bending portion 71 is straightened and passes through the second through hole. The bending portion 71 is bent after passing through the second through hole. The flexible intubation scope 8 carries the airbag 6 and the guidewire 7 into the airway, the guidewire 7 first probes, and then the flexible intubation scope 8 arrives at the supraglottal position. Thereafter, the airbag 6 is deflated through the tube 61, and the flexible intubation scope 8 carries the airbag 6 and the guidewire 7 and removes it from the airway stent assembly.

Compared with the prior art, the airbag 6 of the guidance device provided by the present disclosure can guide the airway stent assembly to be placed in the supraglottis position, and the flexible intubation scope 8 is coaxial with the airbag 6, so that in the process of guiding the airway stent assembly, the airway stent assembly is coaxial with the flexible intubation scope 8, the airway stent assembly no longer deviates from the axis of the flexible intubation scope 8, the advance is hindered by eliminating the aperture difference, and the guidance of the flexible intubation scope 8 to the airway stent assembly is convenient. The recessed portion makes the contact area between the airbag 6 and the airway in the process of guiding the airway stent assembly be reduced, and facilitates assisted ventilation; The guidewire 7 is used to guide the flexible intubation scope 8 into the glottis. The bending portion 71 makes one end of the guidewire 7 have a larger area when advancing in the larynx, and when the guidewire 7 touches the larynx or the inner wall of the trachea, the pressure on the larynx or the inner wall of the trachea is reduced, the injury to the larynx or the trachea is reduced, and the patient safety is ensured; The airway stent assembly of the difficult airway ventilation device can quickly relieve upper airway obstruction and establish a spacious endotracheal intubation access in difficult airway conditions; The gap of the grid structure of the stent 4 can expose the airway and larynx, facilitate the observation of the airway and larynx, assist intubation, sampling or treatment, and also can establish an effective suction channel, and can quickly draw the secretions or blood in the patient's oral cavity. The retrievable unit 42 of the stent 4 makes the stent 4 convenient to remove, and reduces the damage to the patient's oral cavity and airway.

The above is only a better embodiment of the present disclosure, but the scope of protection of the present disclosure is not limited to this, and any person skilled in the art of the art can easily think of changes or substitutions within the scope of the technology disclosed in the invention, shall be covered by the scope of protection of the present disclosure.

1. A difficult airway ventilation device, comprising a guidance assembly and an airway stent assembly, and the guidance assembly is used to guide the airway stent assembly;
the guidance assembly comprises, an airbag and the airbag is used for guiding the airway stent assembly;
the airway stent assembly comprises an inner tube, an outer tube and a locking unit, the outer tube is sleeved on the inner tube the locking unit is arranged on the inner tube and the outer tube, the locking unit is used for locking the inner tube and the outer tube, and the airway stent assembly can be used as a path for ventilation and endotracheal intubation.

2. (canceled)

3. The difficult airway ventilation device of claim 1, wherein the air bag is provided with a first through hole, a flexible intubation scope passes through the first through hole, and the air bag can be fixedly connected with the flexible intubation scope through the first through hole.

4. The difficult airway ventilation device of claim 3, wherein an anti-skid layer is arranged on an inner wall of the first through hole.

5. (canceled)

6. The difficult airway ventilation device of claim 1, wherein the guide assembly further comprises a guide wire, and the air bag is provided with a second through hole, the guide wire is movably connected with the air bag through the second through hole, and the guide wire is used to guide the flexible intubation scope and the air bag into the difficult airway.

7. The difficult airway ventilation device of claim 6, wherein the guidewire comprises a bending portion, and the bending portion is arranged at one end of the guidewire, and the bending portion can be bent at will and maintains the shape after bending.

8. The difficult airway ventilation device of claim 1, wherein one end of the outer flange is provided with an outer flange the outer flange protrudes from an outer wall of the outer tube;
wherein the locking unit comprises an inner flange the inner flange is arranged on the inner wall of the locking unit, and an inner diameter of the inner flange is smaller than an outer diameter of the outer flange, and the outer flange and the inner flange cooperate with each other to limit a movement of the locking unit.

9. The difficult airway ventilation device of claim 8, wherein the locking unit further comprises a groove the inner tube comprises a convex ridge the convex ridge is arranged at one end of the inner tube and the groove and the convex ridge are clamped.

10. The difficult airway ventilation device of claim 1, wherein it further comprises a stent, the stent is arranged between the outer tube and the inner tube, and the stent is used for opening the airway and keeping it open.

11. The difficult airway ventilation device of claim 10, wherein a length of the stent is retractable and an inner diameter can be adjusted.

12. The difficult airway ventilation device of claim 10, wherein the stent comprises a supporting body, a retrievable unit and a tail bracing unit the retrievable unit is arranged at one end of the supporting body, and the tail bracing unit is arranged at the other end of the supporting body; and the tail bracing unit forms a hollow blade-shaped.

13. The difficult airway ventilation device of claim 12, wherein the retrievable unit comprises a first support rod a second support rod, a tail ring and a drawbar, the second support rod is two, the first support rod is located between two second support rods, and one end of each of the two second support rods and one end of the first support rod are all connected with the tail ring and the drawbar is connected with the tail ring to retrieve the stent.

14. An airway ventilation device, comprising an inner tube, an outer tube, a stent, a retrievable mechanism and a skeleton, the outer tube is arranged at one end of the inner tube, the stent is arranged in the outer tube, one end of the stent is provided with the retrievable mechanism, and the other end of the stent is provided with the skeleton;
wherein the retrievable mechanism comprises a first support rod, a second support rod, a tail ring and a drawbar, the second support rod is two, the first support rod is located between two second support rods, and one end of each of the two second support rods and one end of the first support rod are all connected with the tail ring, and the drawbar is connected with the tail ring to retrieve the stent; and the skeleton forms a hollow blade shape.

15. The airway ventilation device of claim 14, wherein the stent is composed of a grid structure, and a length of the stent is retractable and an inner diameter can be adjusted.
